(19) United States
(12) Patent Application Publication
(10) Pub. No.: US 2026/0240454 A1
(43) Pub. Date: Aug. 20, 2026

(54) LOAD RECOGNITION METHOD AND DEVICE, AND WORK SUPPORT SYSTEM

(71) Applicant: Hitachi, Ltd., Tokyo (JP)

(72) Inventor: Takehiro NIIKURA, Tokyo (JP)

(73) Assignee: Hitachi, Ltd., Tokyo (JP)

(21) Appl. No.: 18/853,846

(22) PCT Filed: Feb. 22, 2023
(86) PCT No.: PCT/JP2023/006519

§ 371 Date: Oct. 3, 2024

(30) Foreign Application Priority Data
Apr. 15, 2022 (JP) 2022-067646

(51) Int. Cl. A61B 5/11; G06F 3/01

(52) U.S. Cl. CPC A61B 5/11; G06F 3/011

(57) ABSTRACT

It is possible to prevent, by accurately detecting an overload state accumulated in a worker and promoting improvement, a decrease in work efficiency and occurrence of a failure due to fatigue of the worker and working in an unstable posture. In a load recognition device including a communication unit that receives and transmits a signal, and a processing unit that processes the signal received by the communication unit, the processing unit processes signals received by the communication unit from a plurality of posture sensors mounted on a workwear worn by a worker, estimates a posture of the worker, and determines a high load state of the worker based on a temporal change in the estimated posture. The communication unit receives the signals from the plurality of posture sensors mounted on the workwear worn by the worker, and transmits information on the high load state of the worker determined by the processing unit.

### TECHNICAL FIELD

The present invention relates to a load recognition method and device, and a work support system for measuring a work load on a worker to reduce the load on the worker.

### BACKGROUND ART

In order to enable a worker to stably and safely continue work in a production line, it is necessary to estimate a degree of fatigue of the worker caused by a repeatedly applied load, and to cause the worker to take measures for recovering from fatigue when it is estimated that the fatigue has reached a certain level.

PTL 1 describes a method of controlling a drive unit of a production line according to a load on a worker as a method of determining a degree of the load on the worker and taking measures.

### CITATION LIST

### Patent Literature

PTL 1: JP2018-39076A

### SUMMARY OF INVENTION

### Technical Problem

In PTL 1, when the worker performs work of conveying a workpiece W in cooperation with a robot, a control device, which includes a distance image sensor serving as a hardware circuit and a microcomputer that executes a software program for estimating a posture of a human body based on an output of the distance image sensor, is used, and it is determined whether to change a control amount of the drive unit provided in the production line by comparing a distance image acquired from the distance image sensor with a pattern image registered in advance and detecting a temporal change in a posture of the worker during the work.

However, since a body shape and a physique are different for each worker and the change in the posture that appears when fatigue is accumulated due to a load during work is different for each worker, a degree of actual fatigue of the worker cannot be estimated even when compared with the pattern image registered in advance, and there is a possibility that the worker may overlook an overload state.

An object of the invention is to solve the problems in the related art, and to provide a load recognition method and device, and a work support system that can prevent, by accurately detecting an overload state accumulated in a worker and promoting improvement, a decrease in work efficiency and occurrence of a failure due to fatigue of the worker and working in an unstable posture.

### Solution to Problem

In order to solve the above problems, the invention provides a load recognition device including a communication unit that receives and transmits a signal, and a processing unit that processes the signal received by the communication unit. The processing unit processes signals received by the communication unit from a plurality of posture sensors mounted on a workwear worn by a worker, estimates a posture of the worker, and determines a high load state of the worker based on a temporal change in the estimated posture. The communication unit receives the signals from the plurality of posture sensors mounted on the workwear worn by the worker, and transmits information on the high load state of the worker determined by the processing unit.

In order to solve the above problems, the invention provides a load recognition method for recognizing a state of a load on a worker using a load recognition device including a communication unit and a processing unit. The load recognition method includes: receiving, by the communication unit, signals from a plurality of posture sensors mounted on a workwear worn by the worker; estimating a posture of the worker by processing, by the processing unit, the signals received from the plurality of posture sensors, and determining a high load state of the worker based on a temporal change in the estimated posture; and transmitting, from the communication unit, information on the high load state of the worker based on a result determined by the processing unit.

In order to solve the above problems, the invention provides a work support system. The work support system includes: a plurality of posture sensors mounted on a workwear worn by a worker; a load recognition device configured to receive output signals respectively from the plurality of posture sensors, determine a state of a load on the worker wearing the workwear, and transmit a result of the determination; and a reception unit configured to receive the result of the determination transmitted from the load recognition device, and notify the worker wearing the workwear of the result.

### Advantageous Effects of Invention

According to the invention, it is possible to prevent a decrease in work efficiency and occurrence of a failure due to fatigue of a worker and working in an unstable posture. Further, it is also possible to improve a work environment by preventing accumulation of the fatigue of the worker.

### BRIEF DESCRIPTION OF DRAWINGS

FIG. 1 is a block diagram showing a schematic configuration of a work support system according to Embodiment 1 of the invention.

FIG. 2 is a front view of a worker wearing a workwear on which a plurality of posture sensors and a communication unit are mounted.

FIG. 3 is a diagram showing that, in the work support system according to Embodiment 1 of the invention, signals from the plurality of posture sensors mounted on the workwear are input to an upper arm state estimation unit or a waist state estimation unit of a posture estimation unit, and a First Graph of a temporal change in a rotation angle of an upper arm or a Second Graph of a temporal change in a bending angle of a waist is obtained.

FIG. 4A is a side view of the worker showing a state where the worker is working in a state where knees of the worker are stretched and a position of the waist is high, and FIG. 4B is a side view of the worker showing a state where the worker is working in a state where the knees of the worker are bent and the position of the waist is low.

FIG. 5 is a table showing an example of a relationship among a body part, a length, and a weight of the worker.

FIG. 6A is a side view of the worker showing distances in a horizontal direction from the waist to center of gravity positions of a body, an arm, and a head when the worker is working in a state where the knees of the worker are stretched and the position of the waist is high, and FIG. 6B is a side view of the worker showing distances in the horizontal direction from the waist to the center of gravity positions of the body, the arm, and the head when the worker is working in a state where the knees of the worker are bent and the position of the waist is low.

FIG. 7 is a graph showing a state of a temporal change in a moment applied to the waist of the worker due to a change in the posture of the worker, when the same work is repeatedly performed in Embodiment 1 of the invention.

FIG. 8 is a flowchart showing a flow of data processing using a load recognition system of the work support system according to Embodiment 1 of the invention.

FIG. 9 is a front view of a display scene showing an example of attention information displayed on a display screen of an output unit based on notification information in the work support system according to Embodiment 1 of the invention.

FIG. 10 is a diagram showing that, in a work support system according to Embodiment 2 of the invention, signals from a plurality of posture sensors mounted on a workwear are input to a waist state estimation unit or a knee state estimation unit of a posture estimation unit, and a Third Graph of a temporal change in a bending angle of a waist or a Fourth Graph of a temporal change in a bending angle of a knee is obtained.

FIG. 11A is a side view of the worker showing a relationship between a position where a center of gravity of the worker is projected on a floor surface and an intermediate point on an installation surface of both feet of the worker on a floor when the worker is working in a state where the knees of the worker are stretched and a position of the waist is high, and FIG. 11B is a side view of the worker showing a relationship between a position where the center of gravity of the worker is projected on the floor surface and an intermediate point on the installation surface of both feet of the worker on the floor when the worker is working in a state where the knees of the worker are bent and the position of the waist is low.

FIG. 12 is a graph showing a state of a temporal change in a distance between the intermediate point of both feet of the worker and the position where the center of gravity of the worker is projected on the floor surface, due to a change in a posture of the worker when the same work is repeatedly performed in Embodiment 2 of the invention.

FIG. 13 is a flowchart showing a flow of data processing using a load recognition system of the work support system according to Embodiment 2 of the invention.

FIG. 14 is a diagram showing that, in a work support system according to Embodiment 3 of the invention, in addition to the configuration described with reference to FIG. 3 in Embodiment 1, a signal from a pressure sensor provided in an insole of a shoe among signals from a plurality of posture sensors mounted on a workwear is input to a load data processing unit of a posture estimation unit, and a Fifth Graph of a temporal change in a load related to a foot is obtained.

FIG. 15A is a side view of a worker showing distances in a horizontal direction from a waist to center of gravity positions of a body, an arm, a head, and baggage held by hands when the worker is working in a state where knees of the worker are stretched and a position of a waist is high, and FIG. 15B is a side view of the worker showing distances in the horizontal direction from the waist to the center of gravity positions of the body, the arm, the head, and the baggage held by the hands when the worker is working in a state where the knees of the worker are bent and the position of the waist is low.

### DESCRIPTION OF EMBODIMENTS

According to the invention, a magnitude of a physical quantity such as a moment applied to a body part of a worker is estimated, a state of a load applied to the worker is estimated by observing a change in the physical quantity, and when it is determined that the worker is in a high load state, the load applied to the worker is reduced through notification to the worker or control of a control device.

That is, in the invention, attention is paid to the fact that fatigue accumulated in the worker caused by continuing work in a production line appears in a change in a relative position of a plurality of parts of a body of the worker, and a degree of fatigue of the worker is determined based on a degree of the change in the relative position of the plurality of parts of the body of the worker, and attention is given to the worker, so that an accident leading to an industrial accident can be prevented in advance.

Hereinafter, embodiments of the invention will be described in detail with reference to the drawings. Throughout the drawings for showing the embodiments, components having the same functions are denoted by the same reference signs, and the repeated description thereof is omitted in principle.

However, the invention is not to be construed as being limited to the description of the embodiments described below. It will be easily understood by those skilled in the art that the specific configuration can be changed without departing from the spirit or scope of the invention.

### Embodiment 1

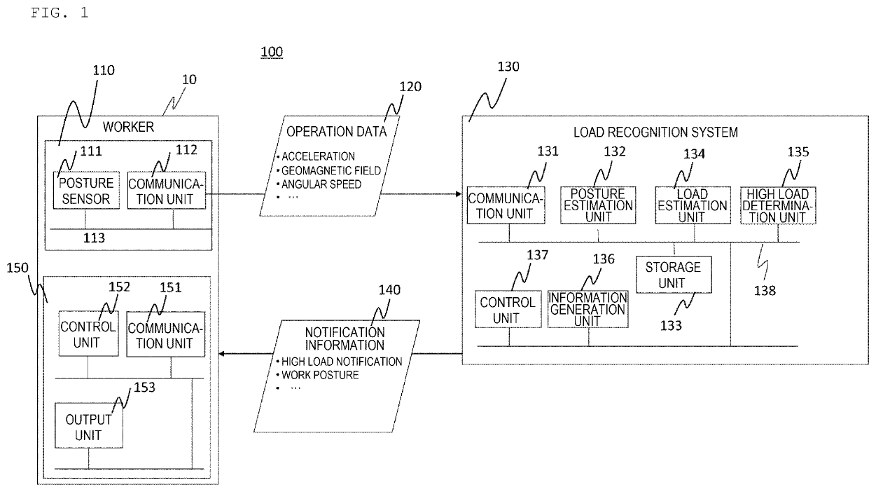

As an embodiment 1 of the invention, a case where work with a relatively small load, such as tightening of a screw, wiring work, and transportation of a lightweight object, is repeatedly executed by a worker in a standing posture will be described with reference to FIGS. 1 to 9.

FIG. 1 shows a configuration of a work support system 100 according to the embodiment.

The work support system 100 according to the embodiment includes a sensor unit 110 mounted on a workwear 10 of a worker, a load recognition system 130 that receives operation data 120 from the sensor unit 110 and determines a state of a load on the worker, and a reception unit 150 that receives notification information 140 generated by the load recognition system 130 and is mounted on the workwear 10 of the worker.

The sensor unit 110 mounted on the workwear 10 of the worker includes a plurality of posture sensors 111, a communication unit 112 that transmits, to the load recognition system 130, the operation data 120 having received output signals from the plurality of posture sensors 111, and a wiring 113 that connects the plurality of posture sensors 111 and the communication unit 112 to one another. The plurality of posture sensors 111 and the communication unit 112 may be connected by wireless communication using Blue Tooth instead of the wiring 113.

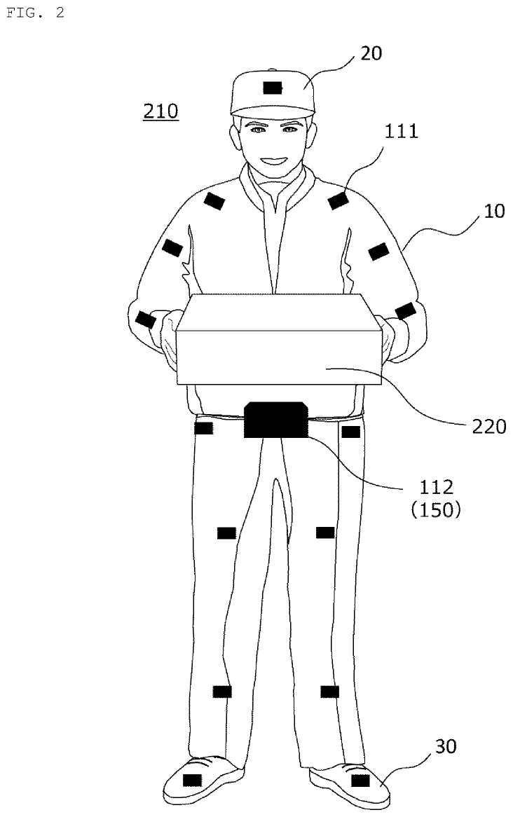

FIG. 2 shows a state where the plurality of posture sensors 111 of the sensor unit 110 and the communication unit 112 are mounted on the workwear 10 of the worker. The posture sensors 111 are also mounted on a plurality of parts of the worker, such as shoulders, arms, a waist, and lower limbs, and a hat 20 and shoes 30. In addition, in FIG. 2, the communication unit 112 is integrated with the reception unit 150.

The posture sensor 111 includes a plurality of sensors such as an acceleration sensor for detecting movements of the shoulder, the arm, the waist, the lower limb, and the head of the worker, a gyro sensor for detecting an inclination, and a geomagnetic sensor for detecting a direction of an operation.

Signals transmitted from the plurality of posture sensors 111 are received by the communication unit 112, and are transmitted from the communication unit 112 to the load recognition system 130 as the operation data 120. The operation data 120 transmitted from the communication unit 112 includes acceleration data detected by the acceleration sensor, inclination data detected by the gyro sensor, and information on the geomagnetic field detected by the geomagnetic sensor in each of the plurality of posture sensors 111. The communication unit 112 and the load recognition system 130 are connected by wireless communication.

As shown in FIG. 1, the load recognition system 130 includes: a communication unit 131 that receives the operation data 120 transmitted from the communication unit 112 of the sensor unit 110 and transmits the notification information 140 to the reception unit 150; a posture estimation unit 132 that receives the operation data 120 received by the communication unit 131, analyzes operation data of the posture sensors 111 mounted on parts of the workwear 10 of the worker, and estimates a posture of the worker; a storage unit 133 that stores data; a load estimation unit 134 that estimates a load applied to, for example, the waist of the worker based on the posture of the worker estimated by the posture estimation unit 132 and the data stored in the storage unit 133; a high load determination unit 135 that determines whether the load applied to the waist of the worker estimated by the load estimation unit 134 is in a high load state for the worker; an information generation unit 136 that generates information on a result of the determination of the high load determination unit 135; and a control unit 137 that controls the whole system. These units are connected by a communication line 138.

The notification information 140 transmitted from the communication unit 131 of the load recognition system 130 to the reception unit 150 includes high load notification information for notifying a state that the load applied to the waist of the worker is in a high load state for the worker, and work posture information for prompting the worker to correct the posture, and the like.

The reception unit 150 includes a communication unit 151 that receives information transmitted from the communication unit 131 of the load recognition system 130, and a control unit 152 that controls an output unit 153 based on a signal received by the communication unit 151 to cause the output unit 153 to display characters and/or images or to transmit a sound or an alarm sound. In addition, an augmented reality (AR) glass and the like may be used as the output unit 153.

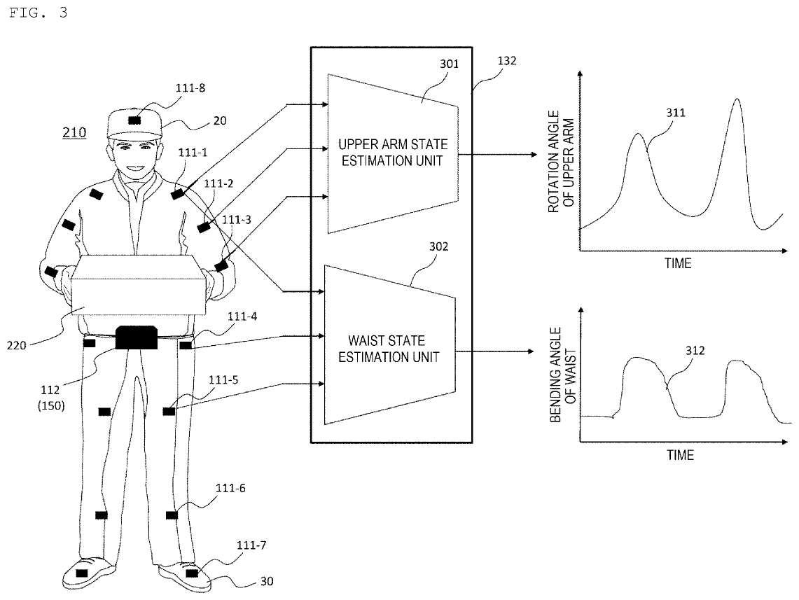

As shown in FIG. 3, signals, which are output from a posture sensor 111-1 mounted on a part of the shoulder of the workwear 10 of the worker, a posture sensor 111-2 mounted on an upper arm portion, and a posture sensor 111-3 mounted on a front arm portion among the plurality of posture sensors 111 mounted on the workwear 10 of the worker, are received, and an upper arm state estimation unit 301 in the posture estimation unit 132 detects a temporal change in a rotation angle of the upper arm as indicated by a curve 311 in a First Graph.

On the other hand, signals, which are output from the posture sensor 111-1 mounted on the part of the shoulder of the workwear 10 of the worker, a posture sensor 111-4 mounted on a waist portion, and a posture sensor 111-5 mounted on a thigh portion among the plurality of posture sensors 111 mounted on the workwear 10 of the worker, are received, and a waist state estimation unit 302 in the posture estimation unit 132 detects a temporal change in a bending angle of the waist as indicated by a curve 312 in a Second Graph.

In addition, a posture sensor 117-7 is mounted on the shoe 30 worn by a worker 210, and a posture sensor 117-8 is mounted on the hat 20 covering the worker 210.

As described above, by using data of the plurality of posture sensors 111 mounted on the workwear 10 of the worker, it is possible to detect a temporal change in a state (posture) such as a position or an inclination angle of each part of the body of the worker.

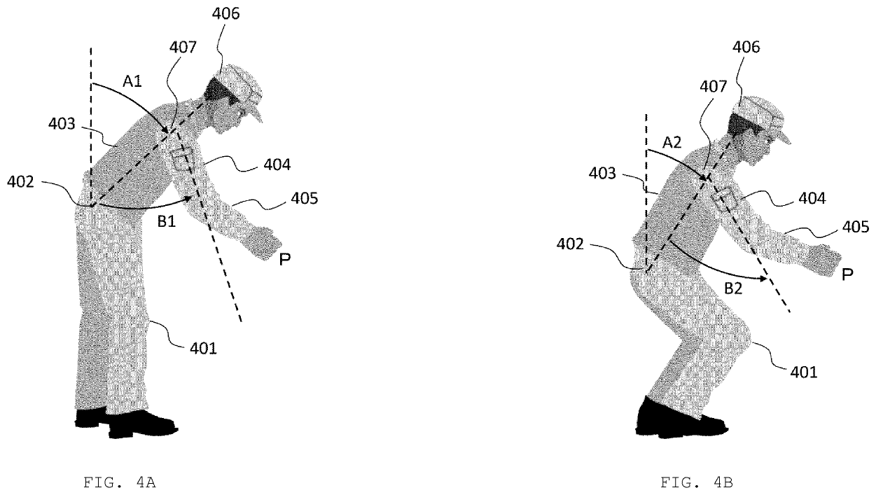

FIGS. 4A and 4B show, as an example of the posture of the worker during work, a state where the worker is working at the same height P in a state shown in FIG. 4A where a knee 401 is stretched to be leaned forward and a state shown in FIG. 4B where the knee 401 is bent to be leaned forward.

For a bending angle A1 from a waist 402 to a body portion 403 with respect to a direction perpendicular to a floor surface in FIG. 4A where the knee 401 is stretched to be leaned forward and an angle B1 of an upper arm 404 with respect to the body portion 403, a bending angle A2 from the waist 402 to the body portion 403 in FIG. 4B where the knee 401 is bent to be leaned forward is smaller than A1, and an angle B2 of the upper arm 404 with respect to the body portion 403 is larger than B1.

As described above, when the worker is working at the same height P, the bending angle from the waist 402 to the body portion 403 and the angle of the upper arm 404 with respect to the body portion 403 are different in FIG. 4A where the knee 401 is stretched to be leaned forward and in FIG. 4B where the knee 401 is bent to be leaned forward. A load (moment) applied to the waist 402 due to weights of a head 406, the body portion 403, the upper arm 404, and a front arm 405 and a load (moment) applied to a shoulder 407 due to the upper arm 404 and the front arm 405 are different.

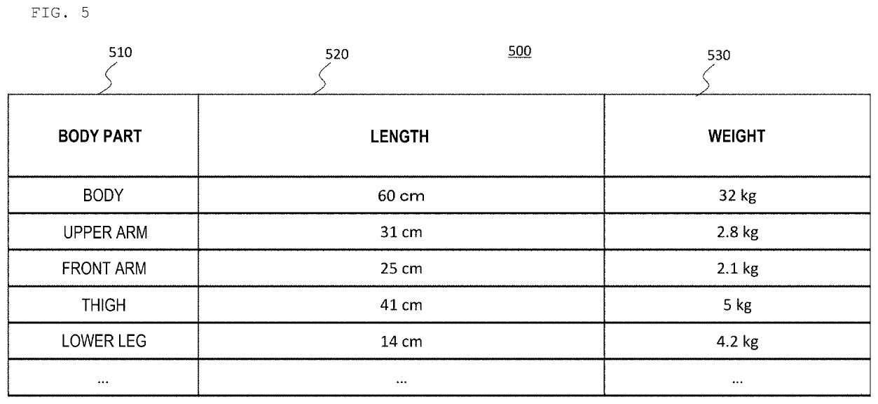

A table 500 of FIG. 5 shows an example of a data set of a length 520 and a weight 530 for each body part 510. FIG. 5 shows an example of a body, an upper arm, a front arm, a thigh, and a lower leg as the body parts 510, and also includes a head and a neck portion.

As described above, the length 520 and the weight 530 are different for each body part 510, and the moment applied to the waist 402 is different according to the posture of the worker.

The data is stored in the storage unit 133 of the load recognition system 130 shown in FIG. 1, and is used when the load estimation unit 134 estimates, for example, the load applied to the waist of the worker.

Although one example of the data set of the length 520 and the weight 530 for each body part 510 of the worker as shown in the table 500 of FIG. 5 is shown, a plurality of data sets including different lengths 520 and weights 530 for each body part 510 may be stored in the storage unit 133, a data set close to the body shape of the worker 210 may be selected from the data sets, and the load estimation unit 134 may estimate, for example, the load applied to the waist of the worker.

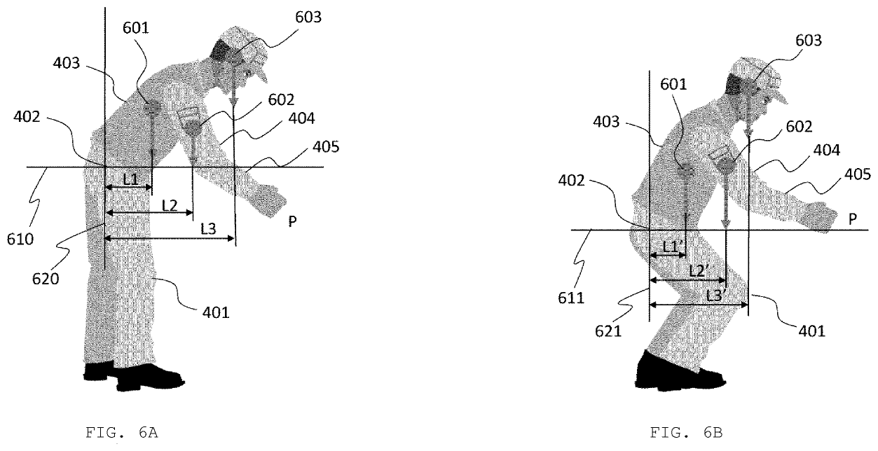

FIG. 6 shows an example of the moment applied to the waist 402 according to the posture of the worker.

In FIG. 6, similarly to FIG. 4, as an example of the posture of the worker during work, a state where the worker is working at the same height P in FIG. 6A where the knee 401 is stretched to be leaned forward and in FIG. 6B where the knee 401 is bent to be leaned forward is shown. Straight lines 610 and 611 represent horizontal lines drawn at height positions of the waist 402, and straight lines 620 and 621 represent vertical lines drawn at positions of the waist 402.

The load applied to the position of the waist 402 of the worker depends on the weight of each of the body portion 403, the upper arm 404, the front arm 405, and the head 406 including the neck portion. In FIGS. 6A and 6B, the weight of the body portion 403 is M1 and a center of gravity position thereof is 601, the weight of the upper arm 404 and the front arm 405 is M2 and a center of gravity position thereof is 602, and the weight of the head 406 is M3 and a center of gravity position thereof is 603.

In FIG. 6A, when a distance from the center of gravity position 601 to the straight line 620 is L1, a distance from the center of gravity position 602 to the straight line 620 is L2, and a distance from the center of gravity position 603 to the straight line 620 is L3, a load (moment) F1 applied to the position of the waist 402 of the worker is expressed as follows.

$$F 1 = M 1 \times L 1 + M 2 \times L 2 + M 3 \times L 3 \qquad ( Equation 1 )$$

On the other hand, in FIG. 6B, when a distance from the center of gravity position 601 to the straight line 621 is L1′, a distance from the center of gravity position 602 to the straight line 621 is L2′, and a distance from the center of gravity position 603 to the straight line 621 is L3′, a load (moment) F2 applied to the position of the waist 402 of the worker is expressed as follows.

$$F 2 = M 1 \times L {1}^{′} + M 2 \times L {2}^{′} + M 3 \times L {3}^{′} \qquad ( Equation 2 )$$

As described with reference to FIGS. 4A and 4B, the bending angle A1 from the waist 402 to the body portion 403 in FIG. 4A where the knee 401 is stretched to be leaned forward is larger than the angle A2 in FIG. 4B where the knee 401 is bent to be leaned forward. Therefore, L1, L2, and L3 in FIG. 6A are larger than L1′, L2′, and L3′ in FIG. 6B, respectively, and as a result, F1 is a value larger than F2.

That is, the moment applied to the waist 402 is larger and a large load is applied to the waist 402 in the state of of FIG. 6A where the knee 401 is stretched to be leaned forward than that in the state of FIG. 6B where the knee 401 is bent to be leaned forward.

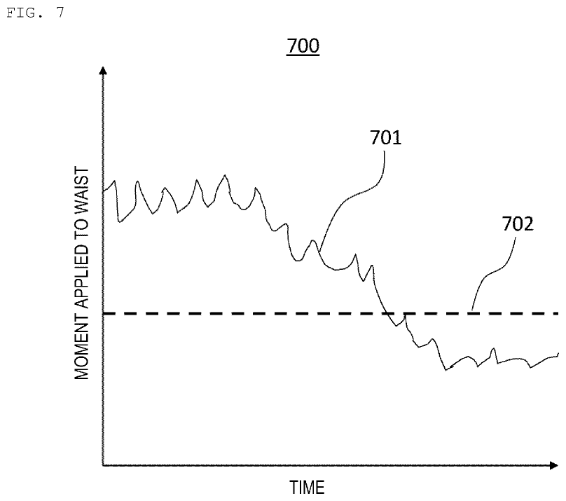

A graph 700 of FIG. 7 shows a state of a temporal change in the moment applied to the waist 402 of the worker due to a change in the posture of the worker when the same work is repeatedly performed.

In the graph of FIG. 7, a moment 701 applied to the waist 402 of the worker is relatively large at an initial stage, but gradually decreases as time elapses. This is estimated to be because the fatigue accumulates due to the load applied to the waist of the worker caused by continuing the same work, therefore the worker changes the posture such that the moment 701 applied to the waist 402 becomes small. That is, the degree of fatigue of the worker can be estimated based on the change in the moment 701 applied to the waist 402 of the worker.

Then, when the moment 701 applied to the waist 402 of the worker is equal to or less than a certain value (in the example shown in FIG. 7, a level indicated by a dotted line 702), by issuing a warning from the load recognition system 130 to the reception unit 150, it is possible to prevent a decrease in work efficiency and occurrence of a failure due to the fatigue of the worker and working in an unstable posture. Further, by preventing the accumulation of the fatigue of the worker, the work environment can be improved.

In the above example, when the moment 701 is equal to or less than the level indicated by the dotted line 702 in FIG. 7, a warning is issued from the load recognition system 130 to the reception unit 150, but the invention is not limited thereto. For example, the change in the moment 701 applied to the waist 402 in FIG. 7 may be represented by a graph obtained by curve approximation, and a warning may be issued from the load recognition system 130 to the reception unit 150 when an inclination of the curve continuously deviates from a preset reference range for a certain time.

Further, in the above example, when the moment 701 is equal to or less than the level indicated by the dotted line 702 in FIG. 7, a warning is issued from the load recognition system 130 to the reception unit 150, but conversely, when the moment 701 applied to the waist 402 is larger than that in the initial state by a certain level or more, a warning may be issued from the load recognition system 130 to the reception unit 150.

Further, when the moment 701 applied to the waist 402 is larger or smaller than the moment in the initial state by a certain level or more (when the moment 701 applied to the waist 402 changes from the moment in the initial state by a certain level or more), a warning may be issued from the load recognition system 130 to the reception unit 150.

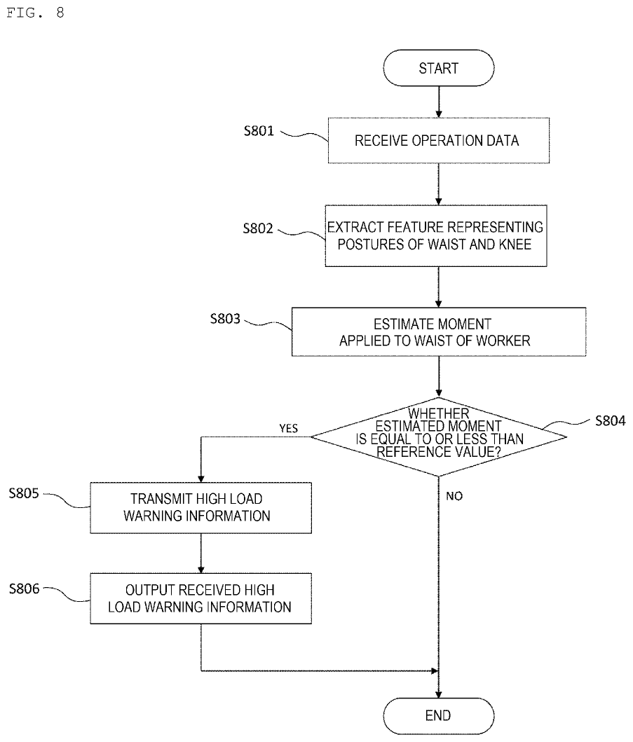

FIG. 8 shows a flow of data processing using the load recognition system 130 in the work support system 100 according to the embodiment. On the premise of the flow of this processing, as shown in FIG. 2, the worker 210 performs work in a state where the workwear 10 on which the plurality of posture sensors 111 of the sensor unit 110, the communication unit 112, and the reception unit 150 are mounted is worn.

First, the communication unit 131 of the load recognition system 130 receives the operation data 120, such as an acceleration, an angular speed, and a geomagnetic field, which is detected by the posture sensor 111 mounted on the workwear 10 worn by the worker and is oscillated from the communication unit 112 (S801).

The data received by the communication unit 131 is transmitted to the posture estimation unit 132 via the communication line 138, and the posture estimation unit 132 extracts features representing the postures of the waist 402 and the knee 401 of the worker 210 that change every moment (S802).

Next, the features representing the postures of the waist 402 and the knee 401 of the worker 210 extracted by the posture estimation unit 132 are transmitted to the load estimation unit 134. The load estimation unit 134 selects a data set close to the body shape of the worker 210 from the plurality of data sets including the features and the lengths 520 and the weights 530 of the body parts 510 as shown in the table 500 of FIG. 5 stored in the storage unit 133, and estimates the moment applied to the waist 402 of the worker 210, which changes every moment, using the data set (S803).

Data on the moment applied to the waist 402 of the worker 210 every moment estimated by the load estimation unit 134 is transmitted to the high load determination unit 135. It is determined whether the estimated moment is equal to or less than a reference value (for example, the dotted line 702 shown in the graph of FIG. 7) (S804).

When the high load determination unit 135 determines that the moment estimated by the load estimation unit 134 is not equal to or less than the reference value (NO in S804), the processing on the operation data received in S801 ends.

On the other hand, when the high load determination unit 135 determines that the moment estimated by the load estimation unit 134 is equal to or less than the reference value (YES in S804), the information is transmitted to the information generation unit 136 to create warning information for notifying the high load state, and the warning information is transmitted as the notification information 140 from the communication unit 131 (S805).

The above is processing executed inside the load recognition system 130.

The notification information 140 transmitted from the communication unit 131 is received by the communication unit 151 of the reception unit 150 mounted on the workwear 10 worn by the worker 210, and the warning information indicating the high load state based on the notification information 140 is output from the output unit 153 (S806).

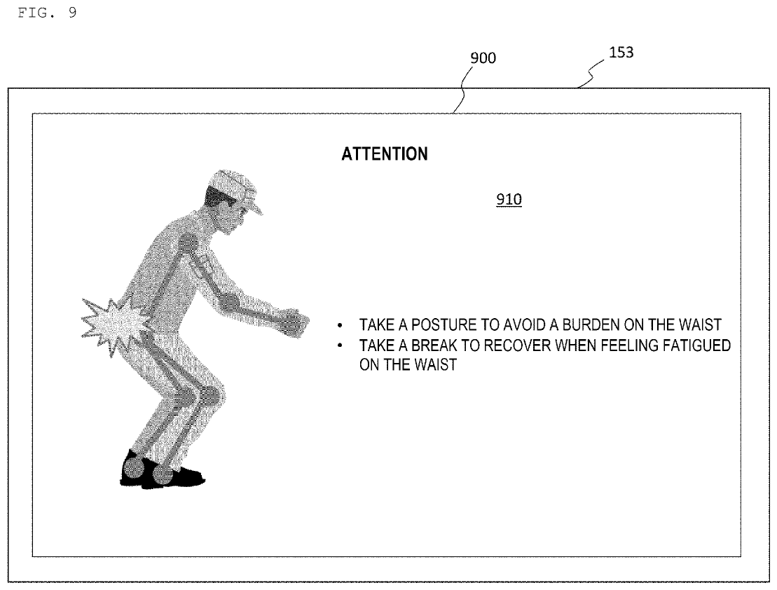

FIG. 9 shows an example of attention information 910 based on the notification information 140 displayed on a display screen 900 of the output unit 153. FIG. 9 shows a case where the display screen 900 displays, as the attention information, information on a change in the posture, such as “take a posture to avoid a burden on the waist”, and information on prompting fatigue recovery, such as “take a break to recover when feeling fatigued on the waist”. As the attention information indicated on the display screen 900, information other than the example shown in FIG. 9 may be displayed.

FIG. 9 shows an example in which the notification information 140 received by the communication unit 151 of the reception unit 150 is output from the output unit 153 and displayed on the display screen 900, but the invention is not limited thereto. For example, information may be transmitted by a sound from a speaker (not shown) provided in the output unit 153. In addition, information may be transmitted as vibration from a vibrator (not shown) provided in the output unit 153. In addition, this information may be combined and transmitted from the output unit 153.

In the above embodiment, a method for estimating the degree of fatigue of the worker based on the change in the moment applied to the waist 402 of the worker 210 has been described, but the invention is not limited thereto. The degree of fatigue of the worker may be estimated based on changes in a moment on a back, a neck, or knees of the worker 210, or the degree of fatigue of the worker may be estimated based on changes in a moment at a plurality of parts.

In addition, in S805, the notification information 140 may be transmitted from the communication unit 131 to a control unit (not shown) that controls a device (not shown) in which the worker 210 is working, in addition to the reception unit 150.

According to the embodiment, it is possible to prevent a decrease in work efficiency and occurrence of a failure due to fatigue of a worker and working in an unstable posture. Further, by preventing the accumulation of the fatigue of the worker, the work environment can be improved.

### Embodiment 2

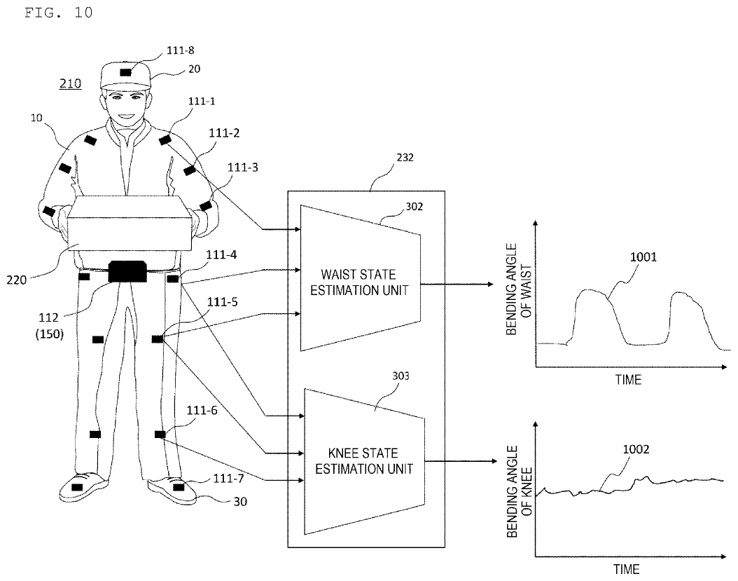

In Embodiment 1, the method for estimating the degree of fatigue of the worker based on the change in the moment applied to the waist 402 of the worker 210 has been described. In the embodiment, a method for estimating fatigue of a worker based on a change in a distance between a center of both feet and a position where a center of gravity of an upper half body is projected will be described with reference to FIGS. 10 to 13.

A configuration of the load recognition system 130 used in the embodiment is basically the same as the configuration described with reference to FIG. 1 in Embodiment 1, but is different in that the posture estimation unit 132 in FIG. 1 is replaced with a posture estimation unit 232 shown in FIG. 10. That is, the posture estimation unit 132 according to Embodiment 1 includes the upper arm state estimation unit 301 and the waist state estimation unit 302 described in FIG. 3, whereas the posture estimation unit 232 according to the embodiment includes the waist state estimation unit 302 and a knee state estimation unit 303 shown in FIG. 10.

As shown in FIG. 10, the posture estimation unit 232 according to the embodiment receives signals which are output from the posture sensor 111-1 mounted on the part of the shoulder of the workwear 10 of the worker, the posture sensor 111-4 mounted on the waist portion, and the posture sensor 111-5 mounted on the thigh portion among the plurality of posture sensors 111 mounted on the workwear 10 of the worker, and the waist state estimation unit 302 in the posture estimation unit 232 detects a temporal change in a bending angle of the waist as indicated by a curve 1001 in a Third Graph.

On the other hand, signals, which are output from the posture sensor 111-4 mounted on the waist portion of the workwear 10 of the worker, the posture sensor 111-5 mounted on a thigh portion, and a posture sensor 111-6 mounted on a lower leg portion among the plurality of posture sensors 111 mounted on the workwear 10 of the worker, are received, and the knee state estimation unit 303 in the posture estimation unit 232 detects a temporal change in a bending angle of the knee as indicated by a curve 1002 in a Fourth Graph.

As described above, by using data of the plurality of posture sensors 111 mounted on the workwear 10 of the worker, it is possible to detect a temporal change in a center of gravity position of the upper half body of the worker 210 based on a change in a state (posture) such as a position or an inclination angle at the waist portion or the knee of the worker.

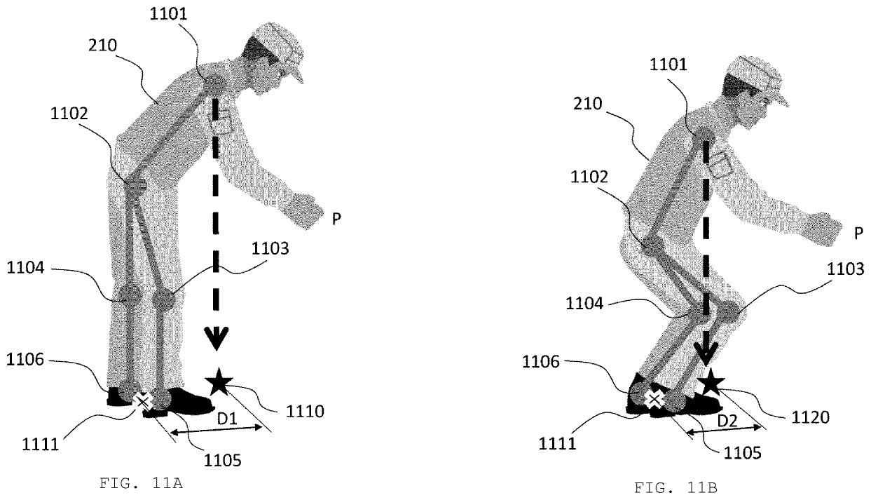

FIGS. 11A and 11B show an example of the change in the distance between the center of both feet and the position where the center of gravity of the upper half body is projected, according to the posture of the worker.

FIGS. 11A and 11B show, as an example of the posture of the worker during work, a state where the worker is working at the same height P in FIG. 11A where a right knee 1103 and a left knee 1104 are stretched to be leaned forward and in FIG. 11B where the right knee 1103 and the left knee 1104 are bent to be leaned forward. 1101 denotes the center of gravity position of the upper half body of the worker 210, 1102 denotes the waist portion of the worker 210, 1103 denotes the right knee, 1104 denotes the left knee, 1105 denotes a right foot's heel, 1106 denotes a left foot's heel, and 1111 denotes a center position between the right foot's heel 1105 and the left foot's heel 1106. In FIGS. 11A and 11B, positions of the right foot's heel 1105 and the left foot's heel 1106 may be the same or different.

In addition, 1110 in FIG. 11A and 1120 in FIG. 11B respectively denote positions where the center of gravity position 1101 of the upper half body of the worker 210 is projected onto a surface (for example, a floor surface) on which the right foot's heel 1105 and the left foot's heel 1106 are placed.

The positions of the right foot's heel 1105 and the left foot's heel 1106 of the worker 210 and postures of the right knee 1103 and the left knee 1104 (bending methods of the right knee 1103 and the left knee 1104) can be obtained by the knee state estimation unit 303 of the posture estimation unit 232 using information on an acceleration, an angular speed, and a geomagnetic field obtained from the signals, which are output from the posture sensor 111-4 mounted on the waist portion of the workwear 10, the posture sensor 111-5 mounted on the thigh portion, and the posture sensor 111-6 mounted on the lower leg portion, and the data of the lengths 520 of the body parts 510 stored in the storage unit 133 as described in FIG. 5. The positions of the right foot's heel 1105 and the left foot's heel 1106 of the worker 210 may be directly obtained from the data of the posture sensor 111-7 mounted on the shoe 30 worn by the worker 210.

In addition, the posture of the waist 1102 is estimated by the waist state estimation unit 302 of the posture estimation unit 232 using the signals output from the posture sensor 111-1 mounted on the part of the shoulder of the workwear 10 of the worker, the posture sensor 111-4 mounted on the waist portion, and the posture sensor 111-5 mounted on the thigh portion.

In the load estimation unit 134, based on information on the positions of the right foot's heel 1105 and the left foot's heel 1106 and information on the postures of the right knee 1103 and the left knee 1104 and the posture of the waist 1102 which are obtained by the posture estimation unit 232, the position (1110 in FIG. 11A and 1120 in FIG. 11B), when the center of gravity position 1101 of the upper half body of the worker is projected onto the floor surface on which the worker 210 stands, and the center position 1111 between the right foot's heel 1105 and the left foot's heel 1106 are obtained, and the distance is calculated. In FIG. 11A, the calculated distance is indicated by D1, and in FIG. 11B, the calculated distance is indicated by D2.

The distance D1 in a state of FIG. 11A where the right knee 1103 and the left knee 1104 are stretched to be leaned forward is larger than the distance D2 in a state of FIG. 11B where the right knee 1103 and the left knee 1104 are bent to be leaned forward, and a load on the worker in the state of FIG. 11A is larger than a load on the worker in the state of FIG. 11B.

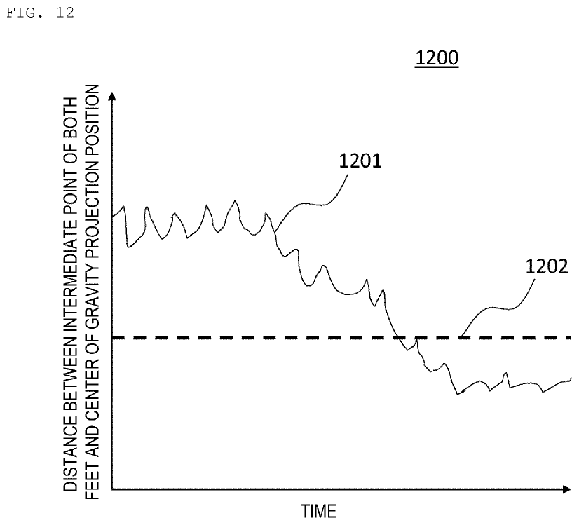

A graph 1200 of FIG. 12 shows a state of a temporal change in a distance 1201 (corresponding to D1 of FIG. 11A or D2 of FIG. 11B), between an intermediate position of both feet of the worker and a position on the floor surface where the center of gravity position of the upper half body is projected, due to a change in the posture of the worker when the same work is repeatedly performed.

In the graph of FIG. 12, the distance 1201, between the intermediate position of both feet of the worker and the position on the floor surface where the center of gravity position of the upper half body is projected, is relatively large at an initial stage, but gradually decreases as time elapses. This is estimated to be because the fatigue accumulates due to the load applied to the waist of the worker caused by continuing the same work, therefore the worker changes the posture.

That is, the degree of fatigue of the worker can be estimated based on the change in the distance 1201 between the intermediate position of both feet of the worker and the position on the floor surface where the center of gravity position of the upper half body is projected.

Then, when the distance 1201, between the intermediate position of both feet of the worker and the position on the floor surface where the center of gravity position of the upper half body is projected, is equal to or less than a certain value (in the example shown in FIG. 12, a level indicated by a dotted line 1202), by issuing a warning from the load recognition system 130 to the reception unit 150, it is possible to prevent a decrease in work efficiency and occurrence of a failure due to the fatigue of the worker and working in an unstable posture. Further, by preventing the accumulation of the fatigue of the worker, the work environment can be improved.

In the above example, when the distance 1201 is equal to or less than the level indicated by the dotted line 1202 in FIG. 12, a warning is issued from the load recognition system 130 to the reception unit 150, but the invention is not limited thereto. For example, the change in the distance 1201, between the intermediate position of both feet of the worker and the position on the floor surface where the center of gravity position of the upper half body is projected, in FIG. 12 may be represented by a graph obtained by curve approximation, and a warning may be issued from the load recognition system 130 to the reception unit 150 when an inclination of the curve continuously deviates from a preset reference range for a certain time.

Further, in the above example, when the distance 1201 is equal to or less than the level indicated by the dotted line 1202 in FIG. 12, a warning is issued from the load recognition system 130 to the reception unit 150, but conversely, when the distance 1201, between the intermediate position of both feet of the worker and the position on the floor surface where the center of gravity position of the upper half body is projected, is larger than that in an initial state by a certain level or more, a warning may be issued from the load recognition system 130 to the reception unit 150.

Further, when the distance 1201, between the intermediate position of both feet of the worker and the position on the floor surface where the center of gravity position of the upper half body is projected, is larger or smaller than that in the initial state by a certain level (or than that in the initial state by a certain ratio) or more (when the distance 1201, between the intermediate position of both feet of the worker and the position on the floor surface where the center of gravity position of the upper half body is projected, is changed by a certain level (ratio) or more from in the initial state), a warning may be issued from the load recognition system 130 to the reception unit 150.

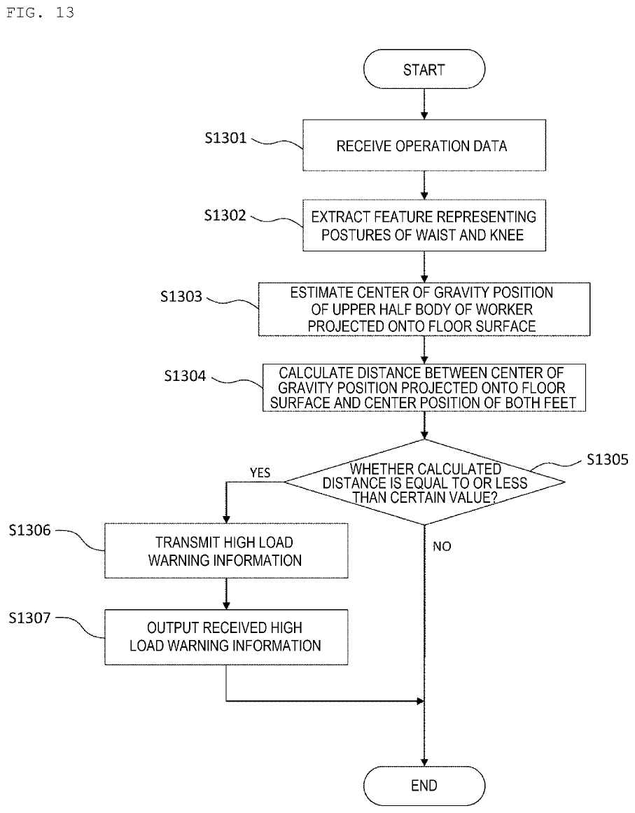

FIG. 13 shows a flow of data processing using the load recognition system 130 in the work support system 100 according to the embodiment. On the premise of the flow of this processing, as shown in FIG. 10, the worker 210 performs work in a state where the workwear 10 on which the plurality of posture sensors 111 of the sensor unit 110, the communication unit 112, and the reception unit 150 are mounted is worn.

First, the communication unit 131 of the load recognition system 130 receives the operation data 120, such as an acceleration, an angular speed, and a geomagnetic field, which is detected by the posture sensor 111 mounted on the workwear 10 worn by the worker and is oscillated from the communication unit 112 (S1301).

The data received by the communication unit 131 is transmitted to the posture estimation unit 132 via the communication line 138, and the posture estimation unit 132 extracts features representing the postures of the waist 402 and the knee 401 of the worker 210 that change every moment (S1302).

Next, the features representing the postures of the waist 402 and the knee 401 of the worker 210 extracted by the posture estimation unit 132 are transmitted to the load estimation unit 134. The load estimation unit 134 selects a data set close to the body shape of the worker 210 from the plurality of data sets including the features and the lengths 520 and the weights 530 of the body parts 510 as shown in the table 500 of FIG. 5 stored in the storage unit 133, and estimates the center of gravity position 1101 of the upper half body of the worker 210 projected on the floor surface, which changes every moment, using the data set (S1303).

The data of the center of gravity position 1101 of the upper half body of the worker 210 projected on the floor surface every moment estimated by the load estimation unit 134 is transmitted to the high load determination unit 135, the distance between the estimated center of gravity position 1101 of the upper half body of the worker 210 projected on the floor surface and the center position 1111 between the foot's heels 1105 and 1106 of the worker 210 is calculated (S1304), and it is determined whether the calculated distance is equal to or less than the reference value (for example, a value indicated by the dotted line 1202 in the graph of FIG. 12, or a certain ratio to a value at the start of measurement) (S1305).

When the high load determination unit 135 determines that the distance calculated in S1304 is not equal to or less than the reference value (NO in S1305), the processing for the operation data received in S1301 ends.

On the other hand, when the high load determination unit 135 determines that the distance calculated in S1304 is equal to or less than the reference value (YES in S1305), the information is transmitted to the information generation unit 136 to create information for notifying the high load state, and the information is transmitted as the notification information 140 from the communication unit 131 (S1306).

The notification information 140 transmitted from the communication unit 131 is received by the communication unit 151 of the reception unit 150 mounted on the workwear 10 worn by the worker 210, and the information based on the notification information 140 is output from the output unit 153 to the display screen 900 as shown in FIG. 9 in Embodiment 1 (S1307).

As a method for outputting the notification information 140 received by the communication unit 151 of the reception unit 150 from the output unit 153, as described in Embodiment 1, for example, information may be transmitted by a sound from a speaker (not shown) provided in the output unit 153. In addition, information may be transmitted as vibration from a vibrator (not shown) provided in the output unit 153. In addition, this information may be combined and transmitted from the output unit 153.

In addition, the posture of the whole body may be estimated and the position of both feet may be estimated without mounting the posture sensors 111 on the shoes 30, or the posture of only the upper half body may be estimated and the position of both feet may be a predetermined value (for example, directly below the position of the waist). Further, a sensor such as a pressure gauge may be disposed on the floor to estimate an intermediate point between both feet based on a pressure distribution.

In the above embodiment, the method for estimating a degree of fatigue of the worker based on a change in the position on the floor surface where the center of gravity position 1101 of the upper half body of the worker 210 is projected has been described, but the invention is not limited thereto. The degree of fatigue of the worker may be estimated based on the change in the center of gravity of the whole body or the position on the floor surface where the center of gravity of the head of the worker 210 is projected, or the degree of fatigue of the worker may be estimated based on the change in the projection positions of the center of gravities of a plurality of parts of the body onto the floor surface.

According to the embodiment, it is possible to prevent a decrease in work efficiency and occurrence of a failure due to fatigue of a worker and working in an unstable posture. Further, by preventing the accumulation of the fatigue of the worker, the work environment can be improved.

### Embodiment 3

As an embodiment 3 of the invention, a case where a weight of an object gripped by a worker is estimated and used for calculation of a magnitude of a moment will be described.

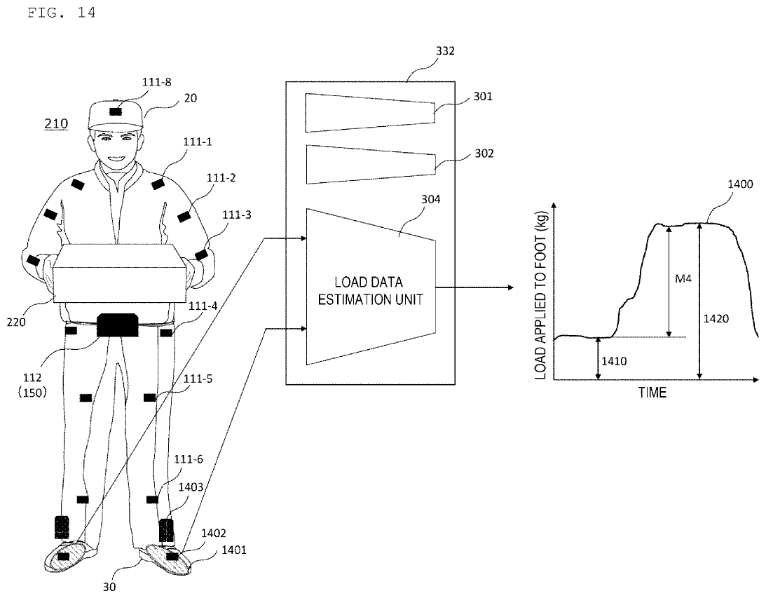

In the embodiment, as shown in FIG. 14, a pressure sensor 1402 is disposed on an insole 1401 of the shoe 30 of the worker 210, a load on baggage lifted by the worker is estimated based on a change in the baggage detected by the pressure sensor 1402. When a fatigue state of the worker described in Embodiment 1 or Embodiment 2 is detected, a weight of the baggage held by the worker is also considered.

The configuration of the load recognition system 130 used in the embodiment is basically the same as the configuration described using FIG. 1 in Embodiment 1, but a posture estimation unit 332 shown in FIG. 14 is different in that a load data estimation unit 304 is added in addition to the upper arm state estimation unit 301 and the waist state estimation unit 302 described in the posture estimation unit 132 of FIG. 1.

That is, the posture estimation unit 132 according to Embodiment 1 uses a method for detecting the fatigue state of the worker 210 based on a change in the posture of the worker 210 including an influence of baggage 220 held by the worker 210 during work, and in the embodiment, the fatigue state of the worker 210 is detected in consideration of a weight of the baggage 220.

In the configuration shown in FIG. 14, the configuration, in which the upper arm state estimation unit 301 receives signals, which are output from the posture sensor 111-1 mounted on a part of the shoulder of the workwear 10 of the worker, the posture sensor 111-2 mounted on the upper arm portion, and the posture sensor 111-3 mounted on the front arm portion, to detect a temporal change in a rotation angle of the upper arm, and the waist state estimation unit 302 receives signals, which are output from the posture sensor 111-1 mounted on the part of the shoulder of the workwear 10 of the worker, the posture sensor 111-4 mounted on the waist portion, and the posture sensor 111-5 mounted on the thigh portion, to detect a temporal change in a bending angle of the waist, as described in FIG. 3 in Embodiment 1 is the same as that in Embodiment 1.

In the embodiment, the load data estimation unit 304 receives an output from the pressure sensor 1402 provided on the insole 1401 of the shoe 30, and detects a load 1400 as shown in a Fifth Graph of FIG. 14 as a load applied to the foot of the worker 210. Here, 1410 is a load applied to the foot of the worker 210 when the worker 210 does not hold the baggage 220, 1420 is a load applied to the foot of the worker 210 when the worker 210 holds the baggage 220, and M4, which is a difference between 1410 and 1420, corresponds to the weight of the baggage 220.

Instead of the pressure sensor 1402 provided on the insole 1401 of the shoe 30, a pressure sheet may be laid in a range in which the worker 210 moves, and the load applied to the foot of the worker 210 may be measured using the pressure sheet.

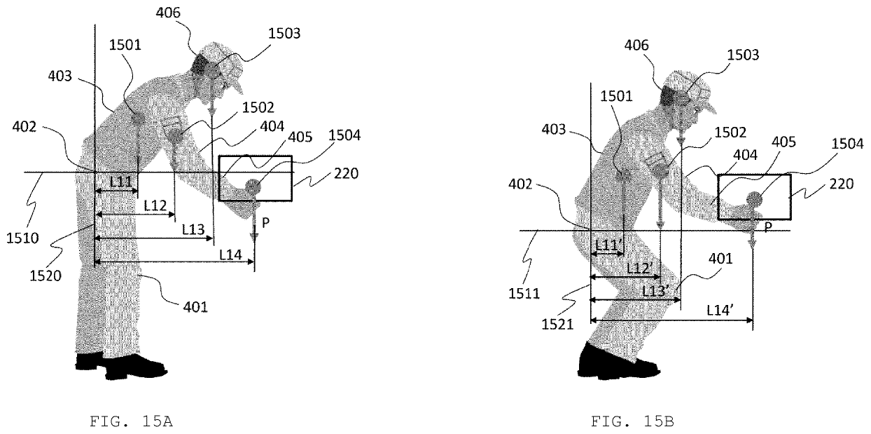

FIG. 15 shows an example of the moment applied to the waist 402 according to the posture of the worker 210.

FIG. 15 shows, as an example of the posture of the worker during work, a state where the worker is holding the baggage 220 and working at the same height P in the state of FIG. 15A where the knee 401 is stretched to be leaned forward and in the state of FIG. 15B where the knee 401 is bent to be leaned forward. Straight lines 1510 and 1511 represent horizontal lines drawn at height positions of the waist 402, and straight lines 1520 and 1521 represent vertical lines drawn at positions of the waist 402.

The load applied to the position of the waist 402 of the worker depends on the weight of each of the body portion 403, the upper arm 404, the front arm 405, and the head 406 including the neck portion, and the weight of the baggage 220. In FIGS. 15A and 15B, the weight of the body portion 403 is M11 and the center of gravity position thereof is 1501, the weight of the upper arm 404 and the front arm 405 is M12 and the center of gravity position thereof is 1502, the weight of the head 406 is M13 and the center of gravity position thereof is 1503, and the weight of the baggage 220 is M14 and the center of gravity position thereof is 1504.

In the state of FIG. 15A, when a distance from a center of gravity position 1501 to the straight line 1520 is L11, a distance from a center of gravity position 1502 to the straight line 1520 is L12, a distance from a center of gravity position 1503 to the straight line 1520 is L13, and a distance from a center of gravity position 1504 to the straight line 1520 is L14, a load (moment) F11 applied to the position of the waist 402 of the worker is expressed as follows.

$$( Equation 3 )$$

$$F 11 = M 11 \times L 11 + M 12 \times L 12 + M 13 \times L 13 + M 14 \times L \times 14$$

On the other hand, in the state of FIG. 15B, when a distance from the center of gravity position 1501 to the straight line 1521 is L11′, a distance from the center of gravity position 1502 to the straight line 1521 is L12′, a distance from the center of gravity position 1503 to the straight line 1521 is L13′, and a distance from the center of gravity position 1504 to the straight line 1521 is L14′, a load (moment) F12 applied to the position of the waist 402 of the worker is expressed as follows.

$$( Equation 4 )$$

$$F 12 = M 11 \times L {11}^{′} + M 12 \times L {12}^{′} + M 13 \times L {13}^{′} + M 14 \times L {14}^{′}$$

As described in Embodiment 1 with reference to FIG. 4, the bending angle A1 from the waist 402 to the body portion 403 in the state of FIG. 4A where the knee 401 is stretched to be leaned forward is larger than the angle A2 in the state FIG. 4B where the knee 401 is bent to be leaned forward. Therefore, L11, L12, and L13 in FIG. 15A are larger than L11′, L12′, and L13′ in FIG. 15B, respectively, and as a result, F11 is a value larger than F12.

That is, the moment applied to the waist 402 is larger and a large load is applied to the waist 402 in the state of FIG. 15A where the knee 401 is stretched to be leaned forward than that in the state of FIG. 15B where the knee 401 is bent to be leaned forward.

Accordingly, as described in Embodiment 1 with reference to the graph of FIG. 7, when the same work is repeatedly performed, the fatigue accumulates due to the load applied to the waist of the worker, and thus the worker changes the posture such that a moment corresponding to the moment 701 applied to the waist 402 decreases. Therefore, the moment applied to the waist 402 of the worker is relatively large at an initial stage but gradually decreases as time elapses. That is, in the embodiment, the degree of fatigue of the worker can also be estimated based on the change in the moment applied to the waist 402 of the worker.

As in the case of Embodiment 1, when the moment corresponding to the moment 701 applied to the waist 402 of the worker is equal to or less than a certain value (in the example shown in FIG. 7, the level indicated by the dotted line 702), by issuing a warning from the load recognition system 130 to the reception unit 150, it is possible to prevent a decrease in work efficiency and occurrence of a failure due to the fatigue of the worker and working in an unstable posture. Further, by preventing the accumulation of the fatigue of the worker, the work environment can be improved.

In addition, in the above example, when the moment is equal to or less than the level indicated by the dotted line 702 in FIG. 7, a warning is issued from the load recognition system 130 to the reception unit 150, but conversely, when the moment corresponding to the moment 701 applied to the waist 402 in FIG. 7 is larger than that in the initial state by a certain level or more, a warning may be issued from the load recognition system 130 to the reception unit 150.

Further, when the moment applied to the waist 402 is larger or smaller than the moment in the initial state by a certain level or more (when the moment applied to the waist 402 changes from the moment in the initial state by a certain level or more), a warning may be issued from the load recognition system 130 to the reception unit 150.

The flow of the data processing using the load recognition system 130 in the work support system 100 according to the embodiment and the attention information 910 based on the notification information 140 displayed on the display screen 900 of the output unit 153 are the same as those described with reference to FIGS. 8 and 9 in Embodiment 1, and thus the description thereof is omitted.

In addition, the embodiment may be combined with the method for estimating the fatigue of the worker based on the change in the distance between the center of both feet and the position where the center of gravity of the upper half body is projected, which is described in Embodiment 2.

According to the embodiment, it is possible to prevent a decrease in work efficiency and occurrence of a failure due to fatigue of a worker and working in an unstable posture. Further, by preventing the accumulation of the fatigue of the worker, the work environment can be improved.

### REFERENCE SIGNS LIST

100: work support system
110: sensor unit
111: posture sensor
112: communication unit
130: load recognition system
131: communication unit
132, 232, 332: posture estimation unit
133: storage unit
134: load estimation unit
135: high load determination unit
136: information generation unit
137: control unit
150: reception unit
151: communication unit
152: control unit
153: output unit
301: upper arm state estimation unit
302: waist state estimation unit
303: knee state estimation unit
304: load data estimation unit

1. A load recognition device comprising:
a communication unit configured to receive and transmit a signal; and
a processing unit configured to process the signal received by the communication unit, wherein
the processing unit processes signals received by the communication unit from a plurality of posture sensors mounted on a workwear worn by a worker, estimates a posture of the worker, and determines a high load state of the worker based on a temporal change in the estimated posture, and
the communication unit receives the signals from the plurality of posture sensors mounted on the workwear worn by the worker, and transmits information on the high load state of the worker determined by the processing unit.

2. The load recognition device according to claim 1, wherein
the processing unit includes
a posture estimation unit that processes the signals from the plurality of posture sensors received by the communication unit and estimates the posture of the worker,
a load estimation unit that estimates a load on the worker based on the posture of the worker estimated by the posture estimation unit,
a high load determination unit that determines the high load state of the worker based on a temporal change in a state of the load on the worker estimated by the load estimation unit, and
an information generation unit that generates information to be notified to the worker when the high load determination unit determines that the worker is in the high load state.

3. The load recognition device according to claim 2, wherein
the posture estimation unit includes
an upper arm state estimation unit that detects a rotation angle of an upper arm of the worker with respect to a shoulder of the worker, and
a waist state estimation unit that detects a bending angle of a waist of the worker.

4. The load recognition device according to claim 2, wherein
the posture estimation unit includes a waist state estimation unit that detects a bending angle of a waist of the worker and a knee state estimation unit that detects a bending angle of a knee of the worker.

5. The load recognition device according to claim 2, wherein
the load estimation unit estimates a load applied to a waist of the worker based on a temporal change in the posture of the worker estimated by the posture estimation unit.

6. A load recognition method for recognizing a state of a load on a worker using a load recognition device including a communication unit and a processing unit, the load recognition method comprising:
receiving, by the communication unit, signals from a plurality of posture sensors mounted on a workwear worn by the worker;
estimating a posture of the worker by processing, by the processing unit, the signals from the plurality of posture sensors received by the communication unit, and determining a high load state of the worker based on a temporal change in the estimated posture; and
transmitting, from the communication unit, information on the high load state of the worker based on a result determined by the processing unit.

7. The load recognition method according to claim 6, wherein
processing the signals from the plurality of posture sensors by the processing unit includes
estimating the posture of the worker by processing the signals from the plurality of posture sensors received by the communication unit,
estimating the load on the worker based on the estimated posture of the worker,
determining the high load state of the worker based on a temporal change in the state of the estimated load on the worker, and
generating information to be notified to the worker when it is determined that the worker is in the high load state.

8. The load recognition method according to claim 7, wherein
estimating the posture of the worker includes
detecting a rotation angle of an upper arm of the worker with respect to a shoulder of the worker,
detecting a bending angle of a waist of the worker, and
estimating the posture of the worker based on the detected rotation angle of the upper arm of the worker and the detected bending angle of the waist of the worker.

9. The load recognition method according to claim 7, wherein
estimating the posture of the worker includes
detecting a bending angle of a waist of the worker,
detecting a bending angle of a knee of the worker, and
estimating the posture of the worker based on the detected bending angle of the waist of the worker and the detected bending angle of the knee of the worker.

10. The load recognition method according to claim 8, wherein
as the estimated load on the worker, a load applied to the waist of the worker is estimated based on the estimated posture of the worker.

11. The load recognition method according to claim 8, wherein
the information on the high load state of the worker transmitted from the communication unit includes information for prompting the worker to recover from fatigue.

12. A work support system comprising:
a plurality of posture sensors mounted on a workwear worn by a worker;
a load recognition device configured to receive output signals respectively from the plurality of posture sensors, determine a state of a load on the worker wearing the workwear, and transmit a result of the determination; and
a reception unit configured to receive the result of the determination transmitted from the load recognition device, and notify the worker wearing the workwear of the result.

13. The work support system according to claim 12, wherein
the load recognition device includes
a communication unit that receives and transmits a signal, and
a processing unit that processes the signal received by the communication unit,
the processing unit processes the signals received by the communication unit respectively from the plurality of posture sensors mounted on the workwear worn by the worker, estimates a posture of the worker, and determines a high load state of the worker based on a temporal change in the estimated posture, and
the communication unit receives the signals from the plurality of posture sensors mounted on the workwear worn by the worker, and transmits information on the high load state of the worker determined by the processing unit.

14. The work support system according to claim 13, wherein
the processing unit includes
a posture estimation unit that processes the signals from the plurality of posture sensors received by the communication unit and estimates the posture of the worker,
a load estimation unit that estimates the load on the worker based on the posture of the worker estimated by the posture estimation unit,
a high load determination unit that determines the high load state of the worker based on a temporal change in the state of the load on the worker estimated by the load estimation unit, and
an information generation unit that generates information to be notified to the worker when the high load determination unit determines that the worker is in the high load state.

15. The work support system according to claim 13, wherein
the reception unit receives the information transmitted from the communication unit, and notifies the worker wearing the workwear by a sound, an image, or both.
